(19) United States
(12) Patent Application Publication
(10) Pub. No.: US 2013/0014755 A1
(43) Pub. Date: Jan. 17, 2013

(54) VAPORIZATION DEVICE

(71) Applicants: Jay Kumar, Yorba Linda, CA (US); Vikram Kumar, Yorba Linda, CA (US)

(72) Inventors: Jay Kumar, Yorba Linda, CA (US); Vikram Kumar, Yorba Linda, CA (US)

(21) Appl. No.: 13/546,696

(22) Filed: Jul. 11, 2012

Related U.S. Application Data
(60) Provisional application No. 61/506,510, filed on Jul. 11, 2011.

(51) Int. Cl. A61M 15/06

(52) U.S. Cl. 128/202.21

(57) ABSTRACT

An apparatus is provided for vaporizing a source of vaporizable substance for delivering an aerosol. The vaporizing apparatus comprises a housing defining a first combustion chamber, a second vaporizing chamber for receiving the vaporizable substance, and a transverse wall spanning the interior of the housing for separating the first chamber and the second chamber. A heat transfer element, comprising an elongated member of heat conductive material, extends from the first chamber through the wall and into the second chamber in heat transfer relation with the vaporizable substance for transferring heat from the first chamber to the vaporizable substance. A gas burner disposed in the combustion chamber converts fuel gas to a flame for heating the combustion chamber and the heat transfer element. The heat transfer element maintains the temperature in the second chamber at or above the vaporization temperature of the substance.

### CROSS-REFERENCES

This application is related to U.S. provisional application No. 61/506,510, filed Jul. 11, 2011, entitled “Vaporizing Cigarette”, naming Jay Kumar and Vikram Kumar as the inventors. The contents of the provisional application are incorporated herein by reference in their entirety, and the benefit of the filing date of the provisional application is hereby claimed for all purposes that are legally served by such claim for the benefit of the filing date.

### BACKGROUND

An aerosol delivery device is described for vaporizing vaporizable matter to produce an aerosol for inhaling and, more particularly, an aerosol delivery device for use as a smoking simulation aerosol delivery device for vaporizing tobacco for pulmonary delivery of nicotine or other active ingredients.

Tobacco and other herbs are smoked by burning in order to release psychoactive constituents, which are then inhaled. However, the burning of such herbs also produces toxins, which largely result from the actual combustion process. Such toxins may be carcinogenic or may result in lung and heart disease. Thus, the smoking of tobacco, herbs and other such plant matter is hazardous and undesirable.

Aerosol delivery devices suitable for vaporizing vaporizable matter without combustion are suitable for use for vaporizing tobacco and other herbs to form an aerosol for inhaling for rapid absorption into the bloodstream. Aerosol delivery devices for delivering vaporized material have several uses, in particular as smoking simulation aerosol delivery devices, or for the delivery of flavorants or therapeutic substances to the user. Various simulated cigarettes or smoking articles wherein a heated aerosol is produced from a carbonaceous external heat source and then delivered to a smoker. In use, a heat source is triggered and the heat produced vaporizes the material, which the user then inhales.

One problem with such aerosol delivery devices is that the exhaust gases of combustion are mixed with the aerosol and inhaled by a user. This is clearly undesirable, since a user is subjected to the products of combustion resulting from the conversion of the fuel gas/air mixture. Also, some aerosol delivery devices first heat ambient air, which the user then inhales through a body of tobacco or nicotine solution. This convective heating method is inferior because the temperature of the tobacco will be variable depending on how fast/hard the user inhales, and because moving air transfers heat less effectively than a stationary heating element that continuously conducts.

In known gas-powered vaporizing aerosol delivery devices, it is difficult to regulate the temperature to which the tobacco is raised such that, in general, it is not possible to prevent the temperature of the tobacco rising to undesirably high temperatures.

For the foregoing reasons, there is a need for a portable aerosol delivery device for vaporizing vaporizable matter from tobacco, an herb or other plant material to produce an inhalable aerosol, in which the temperature of the vaporizable matter may be controlled accurately. The new aerosol delivery device should also segregate the products of combustion from the aerosol produced so that only the aerosol and air is drawn from the aerosol delivery device and inhaled by the user.

### SUMMARY

An apparatus is provided for vaporizing a source of vaporizable substance for delivering an aerosol. The vaporizing apparatus comprises a housing defining a first combustion chamber, a second vaporizing chamber for receiving the vaporizable substance, and a transverse wall spanning the interior of the housing for separating the first chamber and the second chamber. A heat transfer element, comprising an elongated member of heat conductive material, extends from the first chamber through the wall and into the second chamber in heat transfer relation with the vaporizable substance for transferring heat from the first chamber to the vaporizable substance. A gas burner disposed in the combustion chamber converts fuel gas to a flame for heating the combustion chamber and the heat transfer element. The heat transfer element maintains the temperature in the second chamber at or above the vaporization temperature of the substance.

### BRIEF DESCRIPTION OF DRAWINGS

For a more complete understanding of the present invention, reference should now be had to the embodiments shown in the accompanying drawings and described below. In the drawings:

FIG. 1 is a perspective view of an embodiment of a vaporizing device as described herein.

FIG. 2 is an exploded perspective view of the vaporizing device as shown in FIG. 1.

FIG. 3 is a longitudinal cross-section view of the vaporizing device as shown in FIG. 1.

FIG. 4 is a partially exploded perspective view of another embodiment of a vaporizing device as described herein.

### DESCRIPTION

Certain terminology is used herein for convenience only and is not to be taken as a limitation on the invention. For example, words such as “upper,” “lower,” “left,” “right,” “horizontal,” “vertical,” “upward,” and “downward” merely describe the configuration shown in the FIGs. Indeed, the components may be oriented in any direction and the terminology, therefore, should be understood as encompassing such variations unless specified otherwise.

Referring now to the drawings, wherein like reference numerals designate corresponding or similar elements throughout the several views, there is shown an aerosol delivery device for delivering an aerosol of vaporized material to a user, indicated generally by the reference numeral 10. In one embodiment, the aerosol delivery device 10 is adapted for vaporizing constituents in tobacco. In this embodiment, the aerosol delivery device 10 comprises a flame-powered, portable, tubular heater for heating the tobacco to a temperature that is above the vaporizing temperature of tobacco, but below the combustion temperature of the tobacco. The configuration and arrangement of the components of the aerosol delivery device 10 allow for control of the heating of the tobacco within an effective temperature range for vaporization. As will be described below, the aerosol delivery device 10 provides continuous vaporization for an extended period up to, for example, about 10 minutes, or for as long as there is vaporizable material. The aerosol delivery device 10 thus delivers active chemicals, such as nicotine or other herbal or chemical ingredients, to the lungs of the user in a safe, visible vapor form absent a majority of carcinogens found in conventional burning cigarettes.

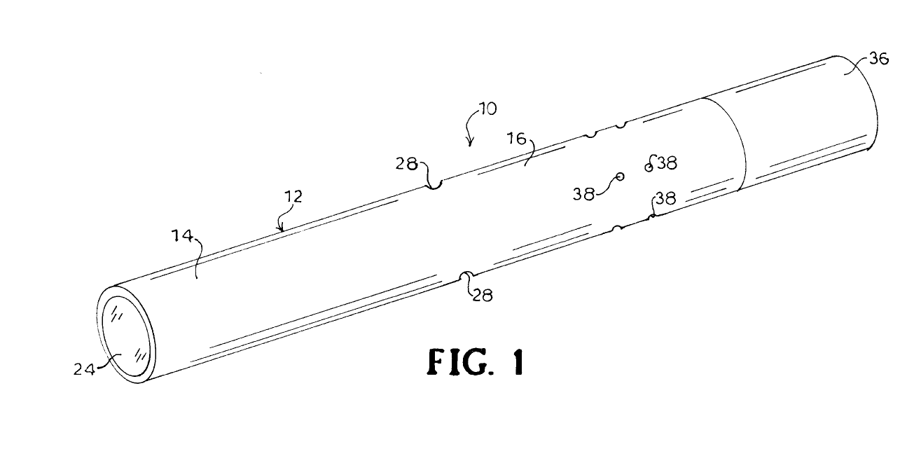

As shown in FIGS. 1-3, the aerosol delivery device comprises a housing 12 including a heat delivery component 14 and a vaporization component 16 for accommodating a source of vaporizable material in heat transfer communication with the heat delivery component 14. The housing 12 is an elongated generally cylindrical member defining a central longitudinal axis. The heat delivery component 14 and the vaporization component 16 are generally axially aligned with each other. As such, the heat delivery component 14 and the vaporization component 16 define respective central axes coinciding with the main central longitudinal axis of the housing 12.

The housing 12 is primarily formed of heat conductive material, which in one embodiment is aluminum. The outer surface of the housing 12 may further comprise a longitudinally-extending outer tube or wrap of substantially non-heat conducting and non-combustible material. The outer surface of the housing is preferably silica aerogel or some other form of aerogel. Other suitable materials include a high heat resistant plastics material of which numerous are commercially available, for example polytetrafluorethylene (PTFE), perfluoroalkoxyethylene (PFA) or a linear aromatic thermoplastic polymer such as that sold by Victrex plc under the trade mark PEEK. Other suitable materials for the outer surface of the housing 12 include ceramic, meerschaum, paper, paperboard, reconstituted tobacco, wood, bamboo, glass, silica, or other durable material with low heat conductivity, and combinations thereof. Any of the foregoing materials may be treated to prevent combustion. Chemical treatments for reducing a propensity for combustion are well known in the art. An insulating outer surface also causes the heat generated to be further contained within the housing 12 and adds an additional variable to the temperature control.

The housing 12 is described and shown in a cigarette-shaped configuration. The length and outer diameter of the housing 12 may also be similar to a cigarette, which are about 3 inches and about 5/16 inches, respectively. It is understood that a housing of any other suitable size and shape and construction may be used. For example, the housing may have a cigar-shaped or a pipe-shaped configuration.

The heat delivery component may be formed of any metallic, ceramic, polymeric, or natural material well known in the art and that is able to withstand the heat of operation of the aerosol delivery device. For example, the heat delivery component may be formed of heat conductive metals such as aluminum, copper, brass, steel, or other metallic alloys, or composite materials.

The heat delivery component 14 comprises a generally cylindrical hollow tube defining a combustion chamber 22 between a proximal end 18 and a distal end 20. A gas burner 24 is located at the proximal end 18 of the combustion chamber 22 for converting a fuel gas/air mixture to a flame for heating the combustion chamber 22 and the housing 12. The flame 26 generated is, in one embodiment, a stable, pre-mixed flame that will not bend due to the orientation of the housing 12. The fuel may be any combustible material with a vapor pressure greater than one atmosphere at the temperature of use. The fuel may include hydrogen, carbon monoxide, or a low molecular weight hydrocarbon, such as methane, ethane, propane, butane, acetylene, or mixtures thereof. In one embodiment, a rechargeable fuel gas reservoir with a refill valve attached thereto may be provided with the heat delivery component 14 for storing fuel in liquid form. The burner 24 is in flow communication with the fuel gas reservoir. An integrated premix-type butane tank and burner is suitable.

An igniter is provided for igniting the fuel gas/air mixture upon activation. Using a piezo-ignition, a flint ignition, or using an external lighter ignition, the butane burner 24 will produce a flame 26. In the case of an external lighter ignition, a small ignition hole (not shown) may be formed in the side wall of the housing 12 and opening into the combustion chamber 22 to allow the external lighter to ignite the burner 24. The flame 26 will burn using only the pre-mix air. Alternatively, the heat delivery component 14 of the housing 12 can be porous or otherwise allow outside air to combine with pre-mix air and with the butane gas in the burner 24 prior to combustion. The degree of aeration of the butane gas upon combustion adds another variable to the temperature regulation—a more premixed flame will be hotter and increase the temperature profile, a less premixed flame can be used to prevent overheating.

Suitable burners for use in the aerosol delivery device 10 are described and shown in U.S. Pat. No. 6,367,715 for a “Method and Device for Producing an Aerosol”, U.S. Pat. No. 6,827,573 for a “Gas Micro Burner”, and U.S. Pat. No. 7,488,171 for a “Gas Micro Burner”, the contents of all of which are hereby incorporated by reference herein in their entirety.

The heat delivery component 14 defines a plurality of exhaust ports 28 extending through the housing 12 adjacent the distal end 20 of the heat delivery component for venting of combustion gases from the combustion chamber 22. The combustion gases may include unreacted fuel and products of incomplete combustion but, ideally, high premixing will lead to nearly complete combustion of fuel.

The vaporization component 16 may be formed of any metallic, ceramic, polymeric, or natural material well known in the art and that is able to withstand the heat of operation of the aerosol delivery device 10. For example, the vaporization component 16 may be formed of ceramic, brass, steel, other metallic alloys, or composite materials. Higher conducting materials will tend to reduce the temperature of the heat conducting element 44 versus insulating materials because the vaporization component 44 absorbs great amounts of the heat, taking it away from the heating element.

The vaporization component 16 comprises a generally cylindrical hollow tube defining a vaporization chamber 30 for holding a source of vaporizable material 31 between a distal end 32 and a proximal end 34. The vaporization chamber 30 is in heat transfer communication with the heating delivery component 14 and configured to release an aerosol from the source of vaporizable material 31 when heated.

The vaporization chamber 30 is configured to accommodate a vaporizable material 50. Preferably, the aerosol delivery device 10 is used as a simulated cigarette wherein the source of vaporizable material may comprises any smokeable material known in the art, such as tobacco, tobacco substitute or another aerosol-generating material or even cartridge comprising a matrix infused with nicotine. In this embodiment, the tobacco may be provided in any suitable form, including loose tobacco, tobacco flakes, granular tobacco, a tobacco sachet, processed tobacco sheet, or extruded tobacco that has been appropriately prepared as a tobacco substrate for the aerosol delivery device 10. The aerosol delivery device 10 as described herein may also be used to deliver to a user an aerosol from any vaporizable material, whether herb, plant or other matter, as well as medicinal compounds, compositions and constituents and psychoactive compounds, compositions and constituents. These embodiments of the invention will be similar in all respects to the aerosol delivery device 10 described and shown herein. The aerosol delivery device 10 can be adapted as necessary for operating within the vaporization temperature for the material to be vaporized.

In one embodiment, a mouthpiece 36 is provided at the distal end 32 of the vaporization component 16. The mouthpiece 36 facilitates the drawing and inhaling of aerosol from the vaporizing chamber 30. For this purpose, one or more air inlet ports 38 may be provided in the housing 12 and opening into the vaporizing chamber 30 to accommodate air being drawn into the vaporization chamber. This facilitates drawing of the aerosol of the vapors from the vaporization chamber 30 through the mouthpiece 36 as the mouthpiece is drawn on. Air drawn through the air inlet ports 38 entrain vapors released from the vaporizable matter 31 in the vaporization chamber 30 so that the aerosol is drawn through the mouthpiece 36 and into the mouth and lungs of the user.

If desired, the mouthpiece 36 may further comprise a filter element through which the vapor passes or the filter element may be disposed vaporizing chamber 16 upstream of the mouthpiece 36. The filter element may be similar to a conventional cigarette end filter, and is provided for filtering out various constituents from the vapors, and also for causing condensation of the vapors to form the aerosol. The filter element can also function as a heat sink to reduce the temperature of vapors before inhalation, in which case insulating materials, such as cotton or other porous insulating material, may be used. The insulating materials absorb heat from the vapor and, along with ambient air entering directly into the mouthpiece 36 and mixing with the vapor, reduce the vapor temperature immediately prior to inhalation. The mouthpiece may also contain various flavorants and other sensation enhancers that the tobacco vapor may pick up immediately before inhalation.

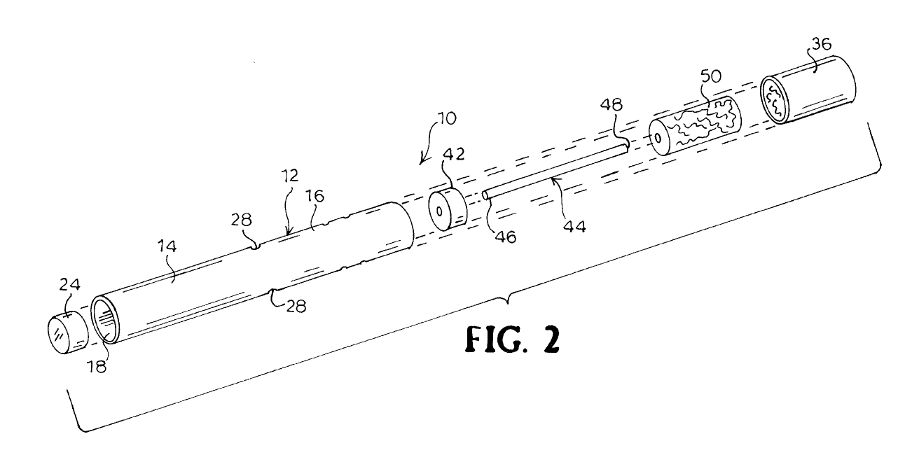

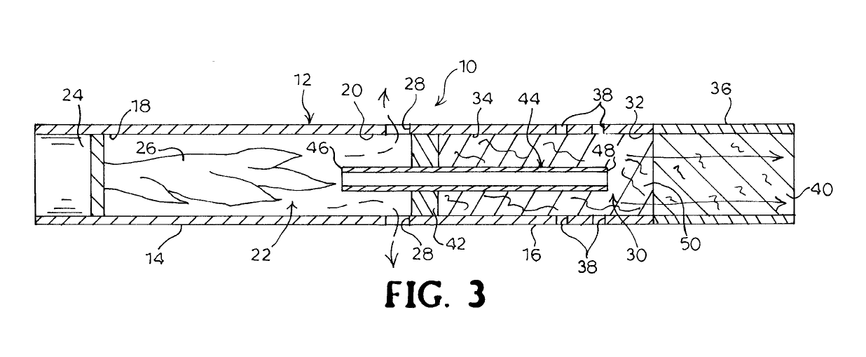

As shown in FIGS. 2 and 3, a wall 42 partitions the interior of the housing 12 extending transversely across the interior of the housing between the combustion chamber 22 and the vaporizing chamber 30. The wall 42 prevents the flame 26 and associated combustion gases generated in the combustion chamber 22 from entering the vaporizing chamber 30. The wall 42 thus shields the vaporizable material from risk of overheating and burning by the flame 26. The wall 42 may be formed of a metallic, ceramic, polymeric material, or the like.

A heat conducting element 44 is provided for effecting heat transfer between the combustion chamber 22 through the primary partition wall and into the vaporizing chamber 30. The heat conducting element 44 is a generally cylindrical hollow tube open at a distal end and formed of heat conductive material similar, such as monel, titanium, aluminum or copper. A proximal end 46 of the heat conducting element 44 extends into the combustion chamber 22 such that the heat conducting element 44 is in direct heat transfer relationship with the flame 26. The heat conducting element 44 is sealed at the proximal end 46. The heat conducting element 44 extends coaxially with the housing 12 and passes through the wall 42 such that the distal end 48 terminates in the vaporizing chamber 30. The wall 42 functions to support the heat conducting element 44 in the housing 12. The wall 42 also helps prevents overheating of the heat conducting element 44 and further adds a temperature control variable based on the thickness of the wall 42. In one embodiment, the inside of the housing 12 including the vaporizing chamber 30 can be lined with a conducting foil, for example aluminum, copper, silver, or gold foil, in order to create a more even heat distribution within the vaporizing chamber 30 between the housing 12 and the heat conducting element 44.

A preferred heat conducting element 44 has an outer diameter of about ( 1/16″) or less. The length is variable between shorter lengths, such as ½″, and longer lengths, such as greater than 3″ The length adds another variable for temperature control, with a longer rod exhibiting a reduced temperature profile. While the heat conducting element 44 is described and shown as a cylindrical hollow tube, it is understood that the heat conducting element 44 can be solid, in which case the outer diameter is preferably tapered toward the distal end 48 to promote more even heat distribution along its length. Alternatively, the heat conducting element 44 can be another shape, such as to improve heat conduction or maximize contact with the vaporizable substance. Further, it should be readily apparent that any number of heat conducting elements 44 may be provided.

The conducting/insulating relationship between the heat delivery component 14 and the heat conducting element 44 is another variable that affects the temperature of the heat conducting element 44. A less conducting heat delivery component 14, such as steel, and a more conducting heat conducting element 44, such as copper, will reach a lower temperature over time than a copper heat delivery component and heat conducting element, so it can be used to prevent overheating, and it also usefully reduces the amount of heat delivered backwards towards the burner.

The aerosol delivery device 10 may be reusable or disposable. In a reusable aerosol delivery device 10, the mouthpiece 36 can be removed to empty the vaporizing chamber 30 of the vaporizable substance. Additionally, the outer surface of the housing 12 could be opened or removed to discard and replace the vaporizable substance, and then rewrapped or otherwise closed.

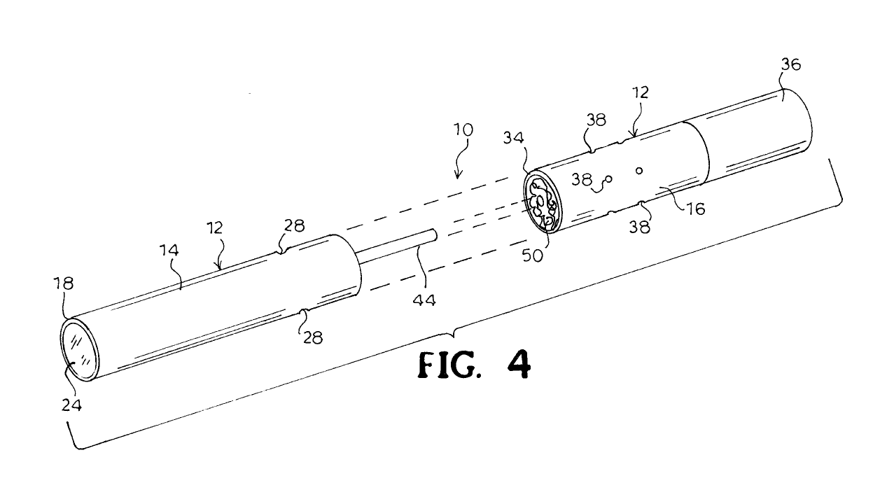

While it is shown that the heat delivery component 14 and the vaporization component 16 may be formed as an integral part of a one-piece housing 12, the heat delivery component 14 and the vaporizing component 16 may be formed as two separate elements of the aerosol delivery device 10, as shown in FIG. 4. The adjacent ends 20, 34 of the heat delivery component 14 and the vaporization component 16 may be complementary in shape and configured such that they are detachably connected for use. In this arrangement, a sealing connection is made, or a sealing means such as an O-ring (not shown) may be provided, for sealing when the vaporization component 16 is fully engaged with the heat delivery component 14. By providing an aerosol delivery device 10 that has detachable, separate components allows the components to be made and sold separately from one another and be either disposable or reusable. For example, the heat delivery component 14, including the fuel tank and burner, combustion chamber and heat conducting element 44, could be removed from the vaporizing component 16 and re-used. The vaporizable material 50 can be removed from the proximal end 34 of the vaporization component 16 and replaced.

In use, tobacco 50 in a suitable form to be vaporized is placed in the vaporization chamber 30. The user then assembles the aerosol delivery device by placing the mouthpiece 36 in the distal end of the vaporization component 16 in one embodiment, or engaging the proximal end 34 of the vaporization component 16 with the heat delivery component 14 in another embodiment, for completing the housing 12 of the aerosol delivery device 10. In the latter assembly step, the distal end 48 of the heat conducting element 44 is inserted into the tobacco 50 in the vaporization chamber 30. A manual on/off valve (not shown) is operated for supplying fuel gas from the reservoir to the burner 24. The fuel is ignited, such as with a piezoelectric igniter which is activated for causing a spark, which in turn ignites the fuel gas/air mixture to burn as a flame 26 in the combustion chamber 22. The flame 26 is in direct contact with the heat conducting element 44 and raises the temperature of the heat conducting element 44 as well as the housing 12. As the heat conducting element 44 is brought up to temperature, the heat is conducted through to the distal end 48 of the heat conducting element 44 where the temperature of the tobacco 50 is raised. The tobacco 50 in the vaporization chamber 30 produces a vapor of the vaporizable components therein. By drawing on the mouthpiece 36 of the vaporization component 16, air enters into the vaporization chamber 30 through the air holes 38 and passes through the vaporization chamber 30. Aerosol from the vapors generated from the tobacco 50 drawn from the vaporization chamber 30 is entrained in the air and drawn through the mouthpiece 36 into the mouth of the user for inhalation.

The aerosol delivery device 10 continues to operate until the on/off switch has been deactivated for extinguishing the burner 24 by isolating the fuel gas from the fuel gas reservoir. When the vaporizable constituents have been exhausted from the tobacco 50, the aerosol delivery device 10 can be disassembled and the used tobacco is removed from the vaporization chamber 30 and replaced with fresh tobacco. As described hereinabove, any of the housing 12, the heat delivery component 14, or the vaporization component 16 can be reusable or disposable. Alternatively, the aerosol delivery device 10 or the components may be used a predetermined number of times and disposed of, in which case the fuel gas reservoir would not be refillable, and would be provided with just sufficient fuel gas for the number of tobacco products or charges of tobacco for which the aerosol delivery device 10 is intended to be used.

Temperature within the aerosol delivery device 10 is controlled by managing the size of the flame 26, the length of the combustion chamber 22 and the distance of the flame from the heat conducting element 44, as well as the overall mass and shape of the heat conducting element. Additionally, once the process of vaporization begins, the rate of temperature increase is reduced as hot vapor carrying heat energy continually flows out of the aerosol delivery device 10. It should be noted that a smaller temperature gradient is a positive for the function of the aerosol delivery device 10. Heat transfer occurs more quickly along the heat conducting element 44, thus providing a smaller temperature gradient along the walls of the heat conducting element and a minimal startup time. With tapering of the heat conducting element 44 the temperature gradient along the length of the heat conducting element can be minimized.

In another aspect, the aerosol delivery device 10 effects a controlled transfer of the heat of a continuous flame 26 to provide temperature ranges appropriate for vaporization, and does so in a manner that fulfills certain functional requirements, such as a minimal start-up time, a maximum time within the vaporizing temperature range, a minimum overall diameter, and a preferred cigarette-like shape and one-handed operation. The aerosol delivery device 10 allows vaporization along the length of the portion of the heat conducting element 44 disposed in the vaporization chamber 30 containing the tobacco, which allows greater quantities of tobacco to be contained and thus vaporized. Moreover, the elongated heat conducting element 44 extends the heating area, which slows the rate of temperature increase and allows the vaporizing temperature range to be maintained for a longer time.

Vaporization at high temperature without combustion leads to a higher volume of vapor, greater nicotine content in the vapor, and larger particle sizes in the vapor compared to lower temperature vaporization, which is critical to delivering the nicotine vapor into the lungs rather than to the mouth and throat membranes as is the case with smaller particle sizes. Research strongly correlates greater delivery of nicotine to the lungs with increased user satisfaction due to the fact that the lung is the most natural method of delivery to the bloodstream, whereas delivery through the upper glands results in much less nicotine reaching the bloodstream.

In one example, an aerosol delivery device uses a 1/16″ diameter, 2″ long copper, hollow heat conducting tube and a ⅜ diameter, 1½″ long aluminum combustion chamber, and a Bernzomatic stock ST-200T pre-mixed burner 0.27″ diameter burner head at a continuous medium-high flame setting. An additive-free brand of cigarettes, American Spirit, is inserted onto the heat conducting element 44. The tobacco begins low-end vaporization about 30 seconds after starting the flame at 250 F and higher-end vaporization at 350 F up to about 420 F. The flame burns continuously during regular operation on a time frame equivalent to smoking a cigarette (about 7 to 10 minutes). Vaporization is also continuous while the tobacco never reaches the 460 F, the temperature at which the tobacco will begin to combust. This example also demonstrates that the aerosol delivery device 10 can be attached to a user's preferred brand of cigarette and thereby create a “vaporizing cigarette” with the cigarette portion being disposable and the heating attachment and other portions being reusable.

The heat conducting element 44 creates an effective “thermal bridge” between the flame 26 and the tobacco 50. This “thermal bridge” distributes the flame heat in such a way that the flame can be maintained during a complete 7-10 minute operation without overheating the tobacco, but simultaneously heats up to vaporization temperatures within a period of time that is less than one minute without changing the butane flow during the entire period of operation. This advantage is realized because our heat conducting element is relatively long and thin, increasing the surface area for heat distribution, which evens out the temperature profile and prevents overheating. A hollow, thin walled configuration decreases heat transfer resistance, allowing the longer tubular heating element to have a fairly even temperature gradient from the proximal end of the tube closest to the flame to the distal end. Given the high heat of the flame, this also minimizes start-up time.

The aerosol delivery device described herein has many advantages, including providing efficient heat delivery to the vaporizable material. This is accomplished by heat transfer by conduction from the combustion chamber to the vaporizing chamber via the heat conduction element extending between the heat delivery and vaporization components. The temperature of the vaporizing chamber is raised and maintained at a minimum temperature for forming the aerosol from desirable vaporizable constituents of tobacco in a controlled way to generate the release of the vaporizable material to the user over a required period while minimizing vaporization of tar and other undesirable constituents of the tobacco.

Although the present invention has been shown and described in considerable detail with respect to only a few exemplary embodiments thereof, it should be understood by those skilled in the art that we do not intend to limit the invention to the embodiments since various modifications, omissions and additions may be made to the disclosed embodiments without materially departing from the novel teachings and advantages of the invention, particularly in light of the foregoing teachings. For example, the aerosol delivery device can be designed to simulate other smoking articles, such as a cigar or a pipe, and can also be formed into different, non-linear, configurations. Accordingly, we intend to cover all such modifications, omission, additions and equivalents as may be included within the spirit and scope of the invention as defined by the following claims. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

We claim:

1. An apparatus for vaporizing a source of vaporizable substance for delivering an aerosol, the vaporizing apparatus comprising:
a housing defining
a first combustion chamber,
a second vaporizing chamber for receiving the vaporizable substance, and
a transverse wall spanning the interior of the housing for separating the first chamber and the second chamber;
a heat transfer element comprising an elongated member of heat conductive material, the heat transfer element extending from the first chamber through the wall and into the second chamber in heat transfer relation with the vaporizable substance for transferring heat from the first chamber to the vaporizable substance; and
a gas burner disposed in the combustion chamber for converting fuel gas to a flame for heating the combustion chamber and the heat transfer element,
wherein the heat transfer element maintains the temperature in the second chamber at or above the vaporization temperature of the substance.

2. A vaporizing apparatus as recited in claim 1, wherein the first chamber and the second chamber are concentric with each other.

3. A vaporizing apparatus as recited in claim 1, wherein the housing defines a plurality of air inlet openings for accommodating air into the second chamber as vapor is drawn from the second chamber.

4. A vaporizing apparatus as recited in claim 1, wherein a proximal end of the heat transfer element is in direct flow communication with the flame generated by the gas burner.

5. A vaporizing apparatus as recited in claim 1, wherein the source of vaporizable material includes tobacco.

6. A vaporizing apparatus as recited in claim 5, wherein the temperature of the vaporizing chamber is maintained at the minimum temperature for forming aerosol from the desirable vaporizable constituents of tobacco.

7. A vaporizing apparatus as recited in claim 1, further comprising a mouthpiece in flow communication with the second chamber for drawing of the aerosol from the vaporizing chamber.
